(19) United States
(12) Patent Application Publication
(10) Pub. No.: US 2015/0191402 A1
(43) Pub. Date: Jul. 9, 2015

(54) PROCESS FOR THE PREPARATION OF OLEFINS

(71) Applicant: INTERNATIONAL RESEARCH MAATSCHAPPIJ B.V., The Hague (NL)

(72) Inventors: Sivakumar Sadasivan Vijayakumari, Gonzales, LA (US); Jeroen Van Westrenen, Amsterdam (NL)

(21) Appl. No.: 14/368,940

(22) PCT Filed: Dec. 20, 2012
(86) PCT No.: PCT/EP2012/076437

§ 371 Date: Jun. 26, 2014

(30) Foreign Application Priority Data
Dec. 27, 2011 (EP) 11195823.7

(51) Int. Cl. C07C 1/20

(52) U.S. Cl. CPC C07C 1/20; C07C 2529/85; C07C 2529/40; C07C 2529/70

(57) ABSTRACT

A process for the preparation of an olefin product, which process comprises the steps of: a) converting a first oxygenate feedstock in an oxygenate-to-olefins conversion system, comprising a first reaction zone in which a first oxygenate feedstock is contacted with a non-zeolitic oxygenate conversion catalyst under first oxygenate conversion conditions, to obtain a first conversion effluent comprising lower olefins and heavy hydrocarbons; b) separating the first conversion effluent into a lower olefin stream and a heavy hydrocarbon stream; and c) feeding the heavy hydrocarbon stream to a separate reactor comprising a second reaction zone in which a second oxygenate feedstock is contacted with a second oxygenate conversion catalyst under second oxygenate conversion conditions, to obtain a second conversion effluent comprising additional lower olefins.

### FIELD OF THE INVENTION

The invention relates to an improved process for preparing lower olefins.

### BACKGROUND

Oxygenate-to-olefin processes are well described in the art. Typically, oxygenate-to-olefin processes are used to produce predominantly ethylene and propylene. An example of such an oxygenate-to-olefin process is described in US Patent Application Publication No. 2011/112344, which is herein incorporated by reference. The publication describes a process for the preparation of an olefin product comprising ethylene and/or propylene, comprising a step of converting an oxygenate feedstock in an oxygenate-to-olefins conversion system, comprising a reaction zone in which an oxygenate feedstock is contacted with an oxygenate conversion catalyst under oxygenate conversion conditions, to obtain a conversion effluent comprising ethylene and/or propylene.

Additional compounds, especially higher molecular weight hydrocarbons are typically produced with the ethylene and propylene in an oxygenate-to-olefins process. A method of improving the yield of lower molecular weight olefins is desired as these olefins, mainly ethylene and propylene, serve as feeds for the production of numerous chemicals.

### SUMMARY OF THE INVENTION

The invention provides a process for the preparation of an olefin product, which process comprises the steps of: a) converting a first oxygenate feedstock in an oxygenate-to-olefins conversion system, comprising a first reaction zone in which a first oxygenate feedstock is contacted with a non-zeolitic oxygenate conversion catalyst under first oxygenate conversion conditions, to obtain a first conversion effluent comprising lower olefins and heavy hydrocarbons; b) separating the first conversion effluent into a lower olefin stream and a heavy hydrocarbon stream; and c) feeding the heavy hydrocarbon stream to a separate reactor comprising a second reaction zone in which a second oxygenate feedstock is contacted with a second oxygenate conversion catalyst under second oxygenate conversion conditions, to obtain a second conversion effluent comprising additional lower olefins.

### BRIEF DESCRIPTION OF THE DRAWINGS

FIG. 1 depicts an embodiment of a process flow scheme in accordance with the invention.

### DETAILED DESCRIPTION

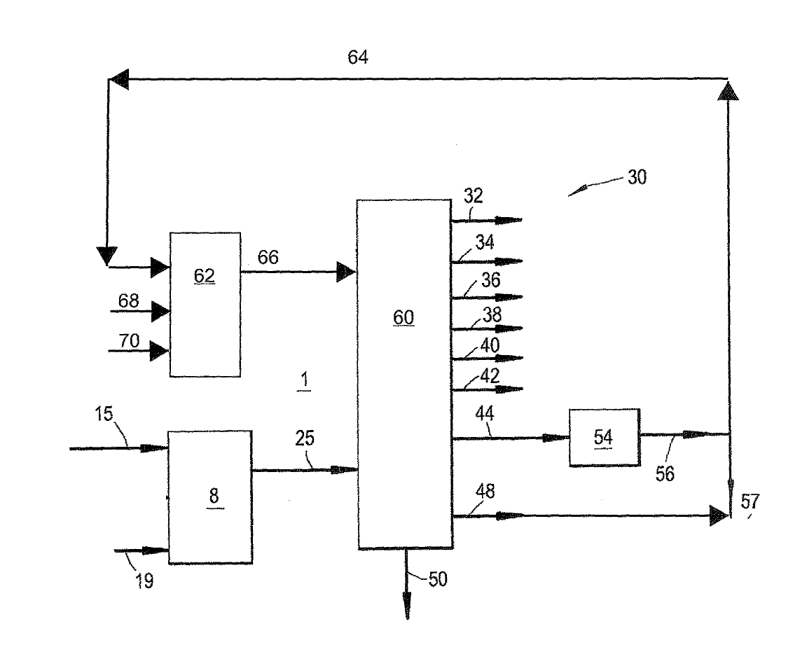

Reference is made to FIG. 1, showing an embodiment of a process flow scheme for an oxygenate-to-olefins conversion process.

The process comprises a first oxygenate-to-olefins (OTO) conversion system 8, a second OTO conversion system 62 and a work-up section 60. An oxygenate feedstock is fed via line 15 to the OTO conversion system 8, for example, comprising methanol and/or dimethylether. An oxygenate feedstock is also fed via line 68 to the OTO conversion system 62. Optionally, a hydrocarbon stream and/or a diluent are fed to the second OTO conversion system 62 via lines 64 or 70, respectively; and a diluent stream 19 may be fed to the first OTO conversion system 8.

In principle every known OTO conversion system and process can be used in conjunction with the present invention, including processes known as Methanol-to-Olefins (MtO) and Methanol to Propylene (MtP). The OTO conversion system and process can for example be as disclosed in US 2005/0038304, incorporated herein by reference; as disclosed in US 2010/206771, incorporated herein by reference; or as disclosed in US 2006/020155 incorporated herein by reference. Other particularly suitable OTO conversion processes and systems with specific advantages are disclosed in US 2009/187058, US 2010/298619, US 2010/268009, US 2010/268007, US 2010/261943, and US 2011/160509, all of which are herein incorporated by reference.

In one embodiment, molecular sieve catalysts are used to convert oxygenate compounds to light olefins. Silicoaluminophosphate (SAPO) molecular sieve catalyst may be used that are selective to the formation of ethylene and propylene. Preferred SAPO catalysts are SAPO-17, SAPO-18, SAPO-34, SAPO-35, SAPO-44, the substituted forms thereof and mixtures thereof. The oxygenate feedstock may comprise one or more aliphatic containing compounds, including alcohols, amines, carbonyl compounds, for example, aldehydes, ketones and carboxylic acids, ethers, halides, mercaptans, sulfides, and the like and mixtures thereof. Examples of suitable feedstocks include methanol, ethanol, methyl mercaptan, ethyl mercaptan, methyl sulfide, methyl amine, di-methyl ether, di-ethyl ether, methyl ethyl ether, methyl chloride, ethyl chloride, dimethyl ketone, formaldehyde, acetaldehyde and various acids such as acetic acid.

In one embodiment, the oxygenate feedstock comprises one or more alcohols having from 1 to 4 carbon atoms and most preferably methanol. The oxygenate feedstock is contacted with a molecular sieve catalyst and is converted to light olefins, preferably ethylene and propylene.

In one embodiment, the first OTO conversion system 8, comprises a non-zeolitic molecular sieve catalyst. This catalyst is preferably a SAPO catalyst, for example, a SAPO-17, SAPO-18, SAPO-34, SAPO-35, SAPO-44, a substituted form thereof or a mixture thereof.

The second OTO conversion system is arranged to receive an olefin stream, and is able to at least partially convert this stream, in particular a stream comprising C₄+ olefins, to ethylene and/or propylene. In one embodiment, the olefin can be contacted with the oxygenate conversion catalyst in the OTO reaction zone; see for example, US 2009/187058, US 2010/298619 and US 2010/268009. The oxygenate conversion catalyst preferably comprises an aluminosilicate, in particular a zeolite.

The first OTO conversion system produces olefins and other hydrocarbons that are passed to the workup section. The light olefins, comprising ethylene and propylene are exported as products. A heavy hydrocarbon stream comprising the hydrocarbons heavier than propane is separated and fed to the second OTO conversion system 62 that produces additional light olefins. A common workup section can be used to separate the various streams produced by both OTO conversion systems.

An olefinic co-feed, preferably a heavy hydrocarbon stream produced by the first OTO conversion system is fed to the second oxygenate-to-olefins conversion system. An olefinic co-feed is a feed containing one or more olefins or a mixture of olefins. The olefinic co-feed may also comprise other hydrocarbon compounds, for example, paraffinic compounds, alkylaromatic compounds, aromatic compounds or mixtures thereof. The olefinic co-feed preferably comprises more than 25 wt % olefins, more preferably more than 50 wt %, still more preferably more than 80 wt % and most preferably in the range of from 95 to 100 wt % olefins. A preferred olefinic co-feed consists essentially of olefins. Non-olefinic compounds in the olefinic co-feed are preferably paraffinic compounds.

The olefins in the olefinic co-feed are preferably mono-olefins. Further, the olefins can be linear, branched or cyclic, but they are preferably linear or branched. The olefins may have from 2 to 12 carbon atoms, preferably 3 to 10 carbon atoms and more preferably from 4 to 8 carbon atoms.

Both OTO processes may be operated in a fluidized bed or moving bed, e.g. a fast fluidized bed or a riser reactor system, and also in a fixed bed reactor or a tubular reactor. A fluidized bed or moving bed, e.g. a fast fluidized bed or a riser reactor system are preferred.

Catalysts suitable for converting the oxygenate feedstock preferably include molecular sieve-comprising catalyst compositions. Such molecular sieve-comprising catalyst compositions typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Molecular sieves preferably have a molecular framework of one, preferably two or more corner-sharing [TO₄] tetrahedral units, more preferably, two or more [SiO₄], [AlO₄] and/or [PO₄] tetrahedral units. These silicon, aluminum and/or phosphorus based molecular sieves and metal containing silicon, aluminum and/or phosphorus based molecular sieves have been described in detail in numerous publications including for example, U.S. Pat. No. 4,567,029. In a preferred embodiment, the molecular sieves have 8-, 10- or 12-ring structures and an average pore size in the range of from about 3 Å to 15 Å.

Suitable molecular sieves are silicoaluminophosphates (SAPO), such as SAPO-17, -18, 34, -35, -44, but also SAPO-5, -8, -11, -20, -31, -36, 37, -40, -41, -42, -47 and −56; aluminophosphates (AlPO) and metal substituted (silico)aluminophosphates (MeAlPO), wherein the Me in MeAlPO refers to a substituted metal atom, including metal selected from one of Group IA, IIA, IB, IIIB, IVB, VB, VIB, VIIB, VIIIB and Lanthanide's of the Periodic Table of Elements, preferably Me is selected from one of the group consisting of Co, Cr, Cu, Fe, Ga, Ge, Mg, Mn, Ni, Sn, Ti, Zn and Zr.

Alternatively, the conversion of the oxygenate feedstock may be accomplished by the use of an aluminosilicate-comprising catalyst, in particular a zeolite-comprising catalyst. Suitable catalysts include those containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48.

Aluminosilicate-comprising catalysts, and in particular zeolite-comprising catalysts, have the additional advantage that in addition to the conversion of methanol or ethanol, these catalysts also induce the conversion of olefins to ethylene and/or propylene. Particular preferred catalyst for this reaction, i.e. converting part of the olefins in the olefinic product, are catalysts comprising at least one zeolite selected from MFI, MEL, TON and MTT type zeolites, more preferably at least one of ZSM-5, ZSM-11, ZSM-22 and ZSM-23 zeolites.

In one preferred embodiment, the molecular sieve in the molecular sieve-comprising catalyst is a non-zeolitic molecular sieve, while part of the olefinic product, in particular at least part of the C₄+ fraction containing olefins, is provided to a subsequent OTO conversion system with a zeolite-comprising catalyst and the C₄+ hydrocarbon fraction is at least partially converted by contact with the zeolite-comprising catalyst.

Preferred catalysts comprise a more-dimensional zeolite, in particular of the MFI type, more in particular ZSM-5, or of the MEL type, such as zeolite ZSM-11. Such zeolites are particularly suitable for converting olefins, including iso-olefins, to ethylene and/or propylene. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. A preferred MFI-type zeolite has a Silica-to-Alumina ratio SAR of at least 60, preferably at least 80.

Particular catalysts include catalysts comprising one or more zeolite having one-dimensional 10-membered ring channels, i.e. one-dimensional 10-membered ring channels, which are not intersected by other channels. Preferred examples are zeolites of the MTT and/or TON type. Preferably, the catalyst comprises at least 40 wt %, preferably at least 50% wt of such zeolites based on total zeolites in the catalyst.

In a particularly preferred embodiment the catalyst comprises in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11.

The catalyst may comprise phosphorus as such or in a compound, i.e. phosphorus other than any phosphorus included in the framework of the molecular sieve. It is preferred that a MEL or MFI-type zeolites comprising catalyst additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05-10 wt % based on the weight of the formulated catalyst. A particularly preferred catalyst comprises MEL or MFI-type zeolites having SAR of in the range of from 60 to 150, more preferably of from 80 to 100, and phosphorus, wherein the phosphorus has preferably been introduced by post-treatment of the formulated catalyst. An even more particularly preferred catalyst comprises ZSM-5 having SAR of in the range of from 60 to 150, more preferably of from 80 to 100, and phosphorus, wherein the phosphorus has preferably been introduced by post-treatment of the formulated catalyst.

It is preferred that molecular sieves in the hydrogen form are used in the oxygenate conversion catalyst in step (g), e.g., HZSM-22, HZSM-23, and HZSM-48, HZSM-5. Preferably at least 50 wt %, more preferably at least 90 wt %, still more preferably at least 95 wt % and most preferably 100 w % of the total amount of molecular sieve used is in the hydrogen form. It is well known in the art how to produce such molecular sieves in the hydrogen form.

Typically the catalyst deactivates in the course of the process, primarily due to deposition of coke on the catalyst. Conventional catalyst regeneration techniques can be employed to remove the coke. It is not necessary to remove all the coke from the catalyst as it is believed that a small amount of residual coke may enhance the catalyst performance and additionally, it is believed that complete removal of the coke may also lead to degradation of the molecular sieve.

The catalyst particles used in the process of the present invention can have any shape known to the skilled person to be suitable for this purpose, for it can be present in the form of spray dried catalyst particles, spheres, tablets, rings, extrudates, etc. Extruded catalysts can be applied in various shapes, such as, cylinders and trilobes. If desired, spent oxygenate conversion catalyst can be regenerated and recycled to the process of the invention. Spray-dried particles allowing use in a fluidized bed or riser reactor system are preferred. Spherical particles are normally obtained by spray drying. Preferably the average particle size is in the range of 1-200 μm, preferably 50-100 μm.

Suitable OTO processes will be further described in detail below. In the first OTO conversion system 8, the oxygenate feedstock is contacted with an oxygenate conversion catalyst under oxygenate conversion conditions, to obtain a conversion effluent comprising lower olefins in line 25. In the second OTO conversion system 62, the oxygenate feedstock, and optionally an olefin co-feed (which can be partly or fully a stream from the first OTO conversion system) are contacted with an oxygenate conversion catalyst under oxygenate conversion conditions, to obtain a conversion effluent comprising lower olefins in line 66. An optional diluent stream may comprise water, steam, inert gases such as nitrogen and/or paraffins, such as methane.

The reaction conditions of the oxygenate conversion include a reaction temperature of 350 to 1000° C., preferably from 350 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar).

Effluents from the OTO conversion systems need to be worked up in order to separate and purify various components as desired, and in particular to separate one or more lower olefin product streams and a heavy hydrocarbon stream. FIG. 1 shows a work-up section 60 which receives and processes at least part of the conversion effluent.

Typically, the effluent is quenched in a quench unit with a quench medium such as water to cool the process gas before feeding it to a compressor. This allows for a smaller compressor and lower power consumption due to reduced gas volume. Any liquid hydrocarbons after the quench are phase separated from liquid water and separately recovered. The water or steam recovered from the quench unit can be partially recycled as diluent to the OTO conversion system via line 19. The water may be treated or purified, for example, to remove catalyst fines or to maintain the pH around neutral.

The vapor components after the quench are typically sent to a compression section, subjected to a caustic wash treatment, dried and sent to a separation including a cold section, to obtain separate streams of the main components. FIG. 1 shows hydrogen stream 32 which may contain some carbon monoxide, light ends stream 34 typically comprising methane and/or carbon monoxide, ethane stream 36, ethylene stream 38, propane stream 40, propylene stream 42, a C₄ stream 44, a C₅₊ stream 48 and a water effluent 50. There can also be a separate outlet for heavy (liquid) hydrocarbons. As known to one of ordinary skill in the art, the work-up section may be designed to provide different purities of each stream, and some of the streams will be produced from the work-up section as combined streams, i.e., C₄, C₅ and C₆ components can be combined. In a preferred embodiment, the C₅ and C₆ streams are combined. Additional reaction, treatment and/or purification steps may be carried out on any of these streams. For example, methane, carbon monoxide and hydrogen may be fed to a methanator to produce methane.

This invention provides for feeding at least part of the various streams from the workup section 60 to the second OTO conversion system 62. Olefins having from 3 to 10 carbon atoms may be fed to the second OTO conversion system 62. The heavy hydrocarbon stream from the first OTO conversion system comprises hydrocarbons having more than 3 carbon atoms or preferably those having 4-6 carbon atoms.

In one embodiment, the two OTO conversion systems share a workup section so the heavy hydrocarbons produced in the second OTO conversion system can be recycled to the second OTO conversion system to produce additional ethylene and propylene. Alternatively, a portion of the heavy hydrocarbon stream can be exported as products.

In another embodiment, the C₄ olefins are recycled to the second OTO conversion system 62, while the C₅ components from both OTO conversion systems are fed to an olefin cracking unit. The products from the olefin cracking unit can then be fed to the combined workup section.

FIG. 1 shows the C₄ stream 44 being fed to a hydrogenation unit 54. All or part of the C₄ stream may be at least partially hydrogenated with a source of hydrogen. The at least partially dehydrogenated C4 stream can be recycled to the OTO conversion system via line 57 and line 17. When recycling to the OTO, the recycle C₄ stream can be a co-feed to the OTO reaction zone or it can be a feed to an optional catalytic olefin cracking zone downstream from the OTO reaction zone. The C4 stream is preferably fed to the second OTO conversion system. Suitable catalysts and conditions are described in U.S. Pat. No. 6,809,227 and US 2004/0102667. Catalysts include those comprising zeolite molecular sieves such as MFI-type, e.g., ZSM-5, or MEL-type, e.g., ZSM-11, as well as Boralite-D and silicalite 2.

In one particular embodiment, the stream 44 comprises a small quantity of di-olefins, in particular butadiene. A small quantity of butadiene is for example, at least 0.01 wt % of butadiene in the stream, in particular at least 0.1 wt %, more in particular at least 0.5 wt. The stream comprising a small quantity of butadiene may be subjected to selective hydrogenation conditions in hydrogenation unit 54 to convert butadiene to butene, but preferably minimizing the hydrogenation of butene to butane. A suitable process for selective hydrogenation is described in U.S. Pat. No. 4,695,560. It is preferred for at least 90 wt % of the butadiene to be converted to butene and less than 10 wt %, preferably less than 5 wt % of the butene to be converted to butane. In another embodiment, the small quantity of butadiene may be left in the stream and recycled to the OTO conversion system.

In one embodiment, C4 olefin streams may be imported from another source and fed to the second OTO conversion system.

The effluent from selective hydrogenation is a C₄ feedstock comprising butene, and butene is a desirable co-feed in OTO reactions, for example in a process in which a catalyst comprising an aluminosilicate or zeolite having one-dimensional 10-membered ring channels and an olefin co-feed is employed. The butene rich effluent can be recycled via line 57.

In one embodiment, the hydrocarbons in the heavy hydrocarbon stream and/or the oxygenates, for example, MTBE will be in a liquid form, and they would need to be heated and vaporized before being fed to the second OTO conversion reactor.

In the OTO processes, some paraffins are formed, for example C₄ saturates that will build up in the system until they are removed. An optional bleed line is present to remove these paraffins from the system.

An article in Studies in Surface Science and Catalysis (2003), 145 and Science and Technology in Catalysis 2002, 109-114 describes an oxygenate to olefins conversion system. The yields of the desired components achieved in the example in this article are shown in Table 1.

TABLE 1

| | Yield |
|---|---|
| Ethylene | 38.74% |
| Propylene | 38.74% |
| Butylene | 11.95% |
| C₅₊ | 4.39% |

### EXAMPLES

### Example 1

Three catalysts, comprising 40 wt % zeolite, 36 wt % kaolin and 24 wt % silica were tested to show their ability to convert 1-butene to an olefinic product. To test the catalyst formulations for catalytic performance, the catalysts were pressed into tablets and the tablets were broken into pieces and sieved.

In the preparation of the first catalyst sample ZSM-23 zeolite powder with a silica to alumina molar ratio (SAR) 46, and ZSM-5 zeolite powder with a SAR of 80 were used in the ammonium form in the weight ratio 50:50. Prior to mixing the powders, the ZSM-5 zeolite powder was treated with phosphorus, resulting in a catalyst that has only one zeolite pre-treated with phosphorus. Phosphorus was deposited on a ZSM-5 zeolite powder with a silica-to-alumina ratio of 80 by means of impregnation with an acidic solution containing phosphoric acid to obtain a ZSM-5 treated zeolite powder containing 2.0 wt % P. The ZSM-5 powder was calcined at 550° C. Then, the powder mix was added to an aqueous solution and subsequently the slurry was milled. Next, kaolin clay and a silica sol were added and the resulting mixture was spray dried wherein the weight-based average particle size was between 70-90 μm. The spray dried catalysts were exposed to ion-exchange using an ammonium nitrate solution. Then, phosphorus was deposited on the catalyst by means of impregnation using acidic solutions containing phosphoric acid (H₃PO₄). The concentration of the solution was adjusted to impregnate 1.0 wt % of phosphorus on the catalyst. After impregnation the catalysts were dried at 140° C. and were calcined at 550° C. for 2 hours. The final formulated catalyst thus obtained is further referred to as catalyst 1.

Another formulated catalyst, catalyst 2, was prepared as described herein above for catalyst 1, with the exception that only ZSM-5 with a SAR of 80 was used and which was not treated with phosphorus prior to spraydrying. The concentration of the phosphorus impregnation solution was adjusted to impregnate 1.5 wt % of phosphorus on the catalyst formulation. The final formulated catalyst thus obtained is further referred to as catalyst 3.

The phosphorus loading on the final catalysts is given based on the weight percentage of the elemental phosphorus in any phosphor species, based on the total weight of the formulated catalyst.

1-Butene in the presence and absence of methanol was reacted over the catalysts which were tested to determine their selectivity towards olefins, mainly ethylene and propylene. For the catalytic testing, a sieve fraction of 60-80 mesh was used. The reaction was performed using a quartz reactor tube of 1.8 mm internal diameter. The molecular sieve samples were heated in nitrogen to the reaction temperature and a mixture consisting of 3 vol % 1-butene, 6% vol % methanol balanced in N₂ was passed over the catalyst at atmospheric pressure (1 bar). In another experiment 3 vol % of 1-butene balanced in N₂ was passed over the catalyst at atmospheric pressure (1 bar).

The Gas Hourly Space Velocity (GHSV) is determined by the total gas flow over the zeolite weight per unit time (ml gas)/(g zeolite·hr). The gas hourly space velocity used in the experiments was 19000 (ml gas)/(g zeolite·hr). The effluent from the reactor was analyzed by gas chromatography (GC) to determine the product composition. The composition was calculated on a weight basis of all hydrocarbons analyzed. The composition was defined by the division of the mass of specific product by the sum of the masses of all products. The effluent from the reactor obtained at several reactor temperatures was analyzed. The results are shown in Table 2.

TABLE 2

| | Methanol | Temp | C₂_{⁼} | C₃_{⁼} | C₄ | C₅ | C₆₊ | LE | C4 sat/C4 |
|---|---|---|---|---|---|---|---|---|---|
| Catalyst | (vol. %) | (° C.) | (wt %) | (wt %) | (wt %) | (wt %) | (wt %) | (wt %) | total (wt/wt) |
| 1 | 0 | 525 | 4.23 | 13.57 | 77.54 | 0.58 | 0.83 | 3.23 | 1.11 |
| 1 | 6 | 525 | 13.59 | 50.66 | 27.37 | 2.47 | 4.31 | 1.60 | 3.70 |
| 2 | 0 | 525 | 8.00 | 23.74 | 63.30 | 0.77 | 1.91 | 2.27 | 2.07 |
| 2 | 6 | 525 | 16.66 | 50.05 | 23.75 | 2.41 | 5.78 | 1.36 | 5.15 |

The experimental results for catalysts 1 and 2 will be considered in understanding the operation of the second OTO conversion system and the additional ethylene and propylene produced by a second OTO conversion system. The C4=conversion for catalyst 1, in the absence of methanol, is 22.46% (calculated as 100-77.54%) at an ethylene plus propylene selectivity of 77.48%. Thus, by combining the yields described in Table 1 with the additional C4=conversion using catalyst 1 in the absence of methanol gives a single pass additional ethylene and propylene yield of 2.08%. Thus combined ethylene plus propylene yield is 79.56% with single pass C4=olefin conversion. In the presence of 2 molar equivalence of methanol relative to 1-butene, C4=conversion increases to 72.63% with ethylene plus propylene selectivity of 88.46%. Thus, combining the yields described in Table 1 with the additional C4=conversion using catalyst 1 in the presence of methanol gives a single pass additional ethylene and propylene yield of 7.65%. Thus combined ethylene plus propylene yield is 85.12% with single pass C4=olefin conversion.

1. A process for the preparation of an olefin product, which process comprises the steps of:
a. converting a first oxygenate feedstock in an oxygenate-to-olefins conversion system, comprising a first reaction zone in which a first oxygenate feedstock is contacted with a non-zeolitic oxygenate conversion catalyst under first oxygenate conversion conditions, to obtain a first conversion effluent comprising lower olefins and heavy hydrocarbons;
b. separating the first conversion effluent into a lower olefin stream and a heavy hydrocarbon stream; and
c. feeding the heavy hydrocarbon stream to a separate reactor comprising a second reaction zone in which a second oxygenate feedstock is contacted with a second oxygenate conversion catalyst under second oxygenate conversion conditions, to obtain a second conversion effluent comprising additional lower olefins.

2. A process as claimed in claim 1 wherein the lower olefin stream consists essentially of propylene and ethylene.

3. A process as claimed in claim 1 wherein the heavy hydrocarbon stream comprises hydrocarbons having a molecular weight greater than the molecular weight of propane.

4. A process as claimed in claim 1 wherein the non-zeolitic oxygenate conversion comprises a molecular sieve selected from the group consisting of silicoaluminophosphates and metal substituted (silico)aluminophosphates

5. A process as claimed in claim 1 wherein the first and second oxygenate feedstocks are the same.

6. A process as claimed in claim 1 wherein the first and second oxygenate feedstocks are selected from the group consisting of methanol, ethanol, tert-alkyl ethers and mixtures thereof.

7. A process as claimed in claim 1 wherein the second oxygenate conversion catalyst comprises a zeolite.

8. A process as claimed in claim 7 wherein the zeolite is selected from the group consisting of ZSM-5, ZSM-11, ZSM-22 and ZSM-23 zeolites and mixtures thereof.
